(19) United States
(12) Patent Application Publication
(10) Pub. No.: US 2010/0049113 A1
(43) Pub. Date: Feb. 25, 2010

(54) COMPRESSION DEVICE FOR USE AFTER VENOUS SURGERY OR ENDOVENOUS TREATMENT OF A LOWER LIMB

(76) Inventor: Jean-Patrick Benigni, Vincennes (FR)

Correspondence Address: Muncy, Geissler, Olds & Lowe, PLLC, P.O. BOX 1364, FAIRFAX, VA, 22038-1364, US

(21) Appl. No.: 12/444,377

(22) PCT Filed: Sep. 27, 2007
(86) PCT No.: PCT/FR07/01581

§ 371 Date: Apr. 3, 2009

(30) Foreign Application Priority Data
Oct. 3, 2006 (FR) 06 08634

(51) Int. Cl. A61F 13/08

(52) U.S. Cl. 602/63; 602/75

(57) ABSTRACT

A compression device for use after venous surgery or endovenous treatment, the device comprising a sleeve (2) of elastic material and having a substantially cylindrical insert (5) suitable for being interposed between the leg and the sleeve (2) in register with a predetermined path.

The present invention relates to a compression device for preventing the formation of a hematoma or an inflammation reaction in the leg of a patient from which, for example, the great saphenous vein or long saphena has been removed, or which has been subjected more generally to equivalent endovenous action (by thermal or chemical action).

### BACKGROUND OF THE INVENTION

Removing this vein is an operation that is widely performed. After the operation, the space left empty by removing the organ from inside the leg can be the seat of an effusion of blood that can lead to a hematoma that is very uncomfortable for the patient.

In order to limit this effusion, in addition to appropriate medical treatment, the leg of the patient is placed in a compression stocking or sleeve that acts from the outside to compress the leg, in general from the ankle to the groin. It is known that in order to compress properly the space left empty by removing the vein (e.g. the saphenous canal) it is appropriate to exert centripetal pressure on the limb of more than 50 hectopascals (40 millimeters of mercury). The degressivity of the pressure associated with the shape of the limb leads to pressure being applied at the ankle that is of at least twice that value, and that is unbearable for the patient.

There exist devices that comprise separate sleeves making it possible to adjust the pressure that is exerted on such and such a portion of a limb (the ankle, the calf, the thigh, . . . ). Nevertheless, such devices are not suitable since it is not at present possible to have a sleeve that compresses the thigh by the amount that is necessary for closing up the saphenous canal.

### OBJECT OF THE INVENTION

The present invention relates to this particular problem of closing up an anatomical space left empty as a result of surgery, and in particular the saphenous canal, or of compressing the great saphenous vein after endovenous treatment.

### SUMMARY OF THE INVENTION

To this end, the invention thus provides a device for compressing a lower limb after venous surgery or endovenous treatment, the device comprising a sleeve of elastic material and including a substantially cylindrical insert suitable for being interposed between the leg and the sleeve or stocking in register with a predetermined path.

In a preferred application, the invention is used after venous surgery, and in particular after “stripping” the great saphenous vein, which makes it necessary to fill in at least in part the saphenous canal. The orthosis of the invention thus comprises a stocking that surrounds the entire leg and an insert for placing in the top portion of this sleeve over the thigh so as to fill in the saphenous canal. The insert thus acts as a “concentrator” of the compression between the stocking (sleeve) and the leg.

Advantageously, at least the surface of the insert that is in contact with the leg is carried by a layer of compressible material. This layer makes contact with the skin of the patient less harsh, thereby increasing comfort for the wearer of the device.

From the structural point of view, the insert of the invention comprises an incompressible central core housed in a compressible sheath. In this embodiment, the insert is reversible since it presents compressible foam or material on at least two opposite faces. The compression device can thus easily be used equally well with either leg of a patient. The section of the insert is preferably oblong with its major axis extending, in service, substantially parallel to the skin of the patient, so as to create a pressure gradient that varies progressively on either side of the longitudinal dimension of the insert.

It should also be observed that, at at least one of its ends, the insert is of decreasing thickness so as to improve the comfort of the patient wearing it, particularly in the vicinity of the knee joint.

Other characteristics and advantages of the invention appear from the following description of an embodiment thereof.

### BRIEF DESCRIPTION OF THE DRAWINGS

Reference is made to the accompanying drawings, in which:

FIG. 1 shows a compression stocking in accordance with the state of the art;

FIG. 2 shows the device of the invention comprising a stocking and an insert;

FIG. 3 is a section view on plane III-III of FIG. 2;

FIG. 4 is a section view of the insert of the device of the invention;

FIG. 5 is a section view on V-V of FIG. 4 showing an end of the insert; and

FIG. 6 is a diagram showing how the insert might be deformed inside the compression stocking of the invention.

### DETAILED DESCRIPTION OF THE INVENTION

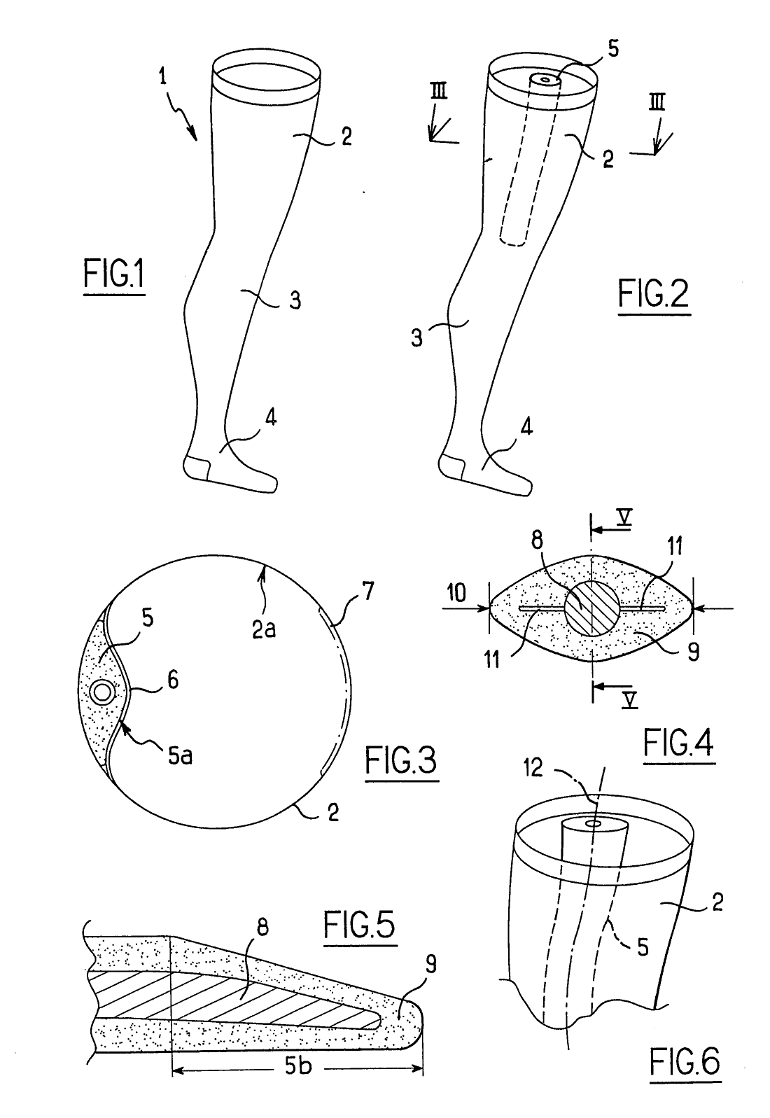

The stocking shown in FIG. 1 is a stocking 1 that is itself known, having a top portion 2 for surrounding the thigh, an intermediate portion 3 for surrounding the calf, and a foot portion 4.

The stocking shown is made as a single piece, however it would not go beyond the ambit of the invention to provide a stocking made up of a plurality of separate sleeves covering the leg after an operation.

At the top portion of this stocking, and as shown in FIG. 2, the invention proposes adding a substantially cylindrical insert 5 over a height that is substantially equal to the height of the patient's thigh, i.e. the height of the top sleeve 2 of the stocking.

As can be seen in the section of FIG. 3, the insert 5 is housed along the inside wall of the sleeve 2 and, if it is the great saphenous vein that has been removed, on the inside of the thigh of a patient, against which it bears via its surface 5a. The insert 5 is secured by any suitable means to the inside surface 2a of the sleeve 2, which means may be holes, a sheath 6, a self-gripping textile 7, . . . . Advantageously, in the device of the invention, the “stocking” portion of the device, includes in its top sleeve 2, two means for fastening the insert that are diametrically opposite each other so that the stocking can be used equally well for the left leg or the right leg of a patient.

The section of the insert 5 shown in FIG. 4 shows one way of making the insert in which an inner core 8 which may be solid or tubular, but which is substantially undeformable in radial compression, is surrounded by a casing of foam 9 that is of almond shape around the core, i.e. it is longer in one dimension than the other. Along this long dimension 10 of the section of the insert 5, the core 8 may include outwardly-projecting fins 11 that stabilize the behavior of the insert in twisting.

FIG. 5 is a section on plane V-V of FIG. 4 showing the insert 5 at one of its ends 5b. It can be seen that at the end the insert, and in particular the inner core 8 of the insert, is of tapering thickness.

Finally, in FIG. 6, there can be seen some of the elements described above bearing the same references. Line 12 represents the longitudinal axis of an insert in accordance with the invention and the shapes that can be given thereto in order to match the position of the insert as a function of the shape of the saphenous canal that is to be filled in. To make it easy to shape the insert, it is preferable to make it with a core 8 that the surgeon can deform easily and permanently by hand along its longitudinal axis 12.

What is claimed is:

1. A compression device for use after venous surgery or endovenous treatment of a lower limb, the device comprising a sleeve of elastic material and including a substantially cylindrical insert suitable for being interposed between the thigh of said lower limb and the sleeve in register with a predetermined path.

2. The device according to claim 1, wherein at least the surface of the insert (5) that is in contact with the thigh is carried by a layer of compressible material.

3. The device according to claim 1, wherein the insert includes an incompressible central core housed in a compressible sheath.

4. The device according to claim 3, wherein the section of the insert is oblong.

5. The device according to claim 4, wherein the central core has two opposite fins extending parallel to the long axis of the oblong section.

6. The device according to claim 1, wherein the material of the core is shapeable in its longitudinal direction.

7. The device according to claim 1, wherein the insert possesses an end portion of decreasing thickness.

8. The device according to claim 1, wherein the elastic sleeve includes holder means for holding the insert to its inside face.

9. The device according to claim 8, wherein the holder means are provided in duplicate along two opposite generator lines of the elastic sleeve.
